SEQUENCE LISTING (3 sequences)

SEQ ID NO: 1 - protein, 219 aa, synthetic construct
MDPAPGVLDPRAAPPALLGTPQAEVLEDVLREQFGPLPQLAAVCRLKRLPSGGYSSTENLQLVLERRRVANAKERERIKNLNRGFARLKALVPFLPQSRKPSKVDILKGATEYIQVLSDLLEGAKDSKKQDPDEQSYSNNSSESHTSSARQLSRNITQHISCAFGLKNEEEGPWADGGSGEPAHACRHSVMSTTEIISPTRSLDRFPEVELLSHRLPQV

SEQ ID NO: 2 - protein, 289 aa, synthetic construct
MTGVFDRRVPSIRSGDFQAPFQTSAAMHHPSQESPTLPESSATDSDYYSPTGGAPHGYCSPTSASYGKALNPYQYQYHGVNGSAGSYPAKAYADYSYASSYHQYGGAYNRVPSATNQPEKEVTEPEVRMVNGKPKKVRKPRTIYSSFQLAALQRRFQKTQYLALPERAELAASLGLTQTQVKIWFQNKRSKIKKIMKNGEMPPEHSPSSSDPMACNSPQSPAVWEPQGSSRSLSHHPHAHPPTSNQSPASSYLENSASWYTSAASSINSHLPPPGSLQHPLALASGTLY

SEQ ID NO: 3 - protein, 270 aa, synthetic construct
MQYPHPGPAAGAVGVPLYAPTPLLQPAHPTPFYIEDILGRGPAAPTPAPTLPSPNSSFTSLVSPYRTPVYEPTPIHPAFSHHSAAALAAAYGPGGFGGPLYPFPRTVNDYTHALLRHDPLGKPLLWSPFLQRPLHKRKGGQVRFSNDQTIELEKKFETQKYLSPPERKRLAKMLQLSERQVKTWFQNRRAKWRRLKQENPQSNKKEELESLDSSCDQRQDLPSEQNKGASLDSSQCSPSPASQEDLESEISEDSDQEVDIEGDKSYFNAG

(19) United States
(12) Patent Application Publication
(10) Pub. No.: US 2025/0207091 A1
(43) Pub. Date: Jun. 26, 2025

(54) METHODS AND COMPOSITIONS FOR PRODUCING PRIMORDIAL GERM CELL-LIKE CELLS

(71) Applicant: President and Fellows of Harvard College, Cambridge, MA (US)

(72) Inventors: Christian Kramme, Cambridge, MA (US); Merrick Pierson Smela, Cambridge, MA (US); Pranam Chatterjee, Cambridge, MA (US); George M. Church, Cambridge, MA (US)

(73) Assignee: President and Fellows of Harvard College, Cambridge, MA (US)

(21) Appl. No.: 18/851,987

(22) PCT Filed: Mar. 30, 2023
(86) PCT No.: PCT/US2023/065143

§ 371 Date: Sep. 27, 2024

Related U.S. Application Data
(60) Provisional application No. 63/326,656, filed on Apr. 1, 2022.

(51) Int. Cl. C12N 5/0735; C07K 14/47

(52) U.S. Cl. CPC C12N 5/0611; C07K 14/4702; C12N 2501/11; C12N 2501/125; C12N 2501/155; C12N 2501/16; C12N 2501/727; C12N 2506/45; C12N 2510/00

(57) ABSTRACT

Provided herein are methods and compositions for differentiating induced pluripotent stem cells into primordial germ cell-like cells by overexpressing transcription factors such as DLX5, HHEX, and/or FIGLA.

### CROSS-REFERENCE TO RELATED APPLICATIONS

This application claims the benefit under 35 U.S.C. § 119(e) of U.S. Provisional Application No. 63/326,656, filed Apr. 1, 2022, which is hereby incorporated by reference in its entirety.

### REFERENCE TO AN ELECTRONIC SEQUENCE LISTING

The contents of the electronic sequence listing (H049870759WO00-SEQ-KVC.xml; Size: 4,453 bytes; and Date of Creation: Mar. 27, 2023) is herein incorporated by reference in its entirety.

### BACKGROUND

Primordial germ cells are germline stem cells that give rise to gametes in vertebrates. Primordial germ cells migrate to the developing gonads where they differentiate into sperm or eggs. Primordial germ cell dysfunction forms the basis of many forms of human female infertility, yet efficient methods for generating primordial germ cells in vitro remain elusive.

### SUMMARY

The present disclosure relates, at least in part, to methods and compositions for generating primordial germ cell-like cells (PGCLCs) in vitro from pluripotent stem cells (PSCs). The present disclosure provides experimental data demonstrating, unexpectedly, that overexpression of certain transcription factors, for example, DLX5, HHEX, and FIGLA, is sufficient to generate PGCLC (e.g., PGCLCs that are NANOS3⁺, SOX17⁺, TFAP2C⁺, PRDM1⁺, OCT4⁺, CD38⁺, EPCAM⁺, ITGA6⁺, and/or SOX2⁻) from PSCs in as few as four days.

Some aspects of the present disclosure provide a PSC comprising: an engineered polynucleotide comprising an open reading frame encoding a protein selected from DLX5, HHEX, and FIGLA.

In some embodiments, the PSC comprises the engineered polynucleotide comprising an open reading frame encoding DLX5.

In some embodiments, the PSC comprises the engineered polynucleotide comprising an open reading frame encoding HHEX.

In some embodiments, the PSC comprises the engineered polynucleotide comprising an open reading frame encoding FIGLA.

In some embodiments, the PSC expresses or overexpresses: DLX5; HHEX; FIGLA; DLX5 and HHEX; DLX5 and FIGLA; HHEX and FIGLA; or DLX5, HHEX, and FIGLA.

In some embodiments, the open reading frame of the engineered polynucleotide is operably linked to a heterologous promoter.

In some embodiments, the heterologous promoter is an inducible promoter.

Other aspects of the present disclosure provide a PSC comprising: a protein selected from DLX5, HHEX, and FIGLA, wherein the protein is overexpressed.

In some embodiments, the PSC expresses or overexpresses: DLX5; HHEX; FIGLA; DLX5 and HHEX; DLX5 and FIGLA; HHEX and FIGLA; or DLX5, HHEX, and FIGLA.

In some embodiments, the PSC is a human PSC.

In some embodiments, the PSC is an induced PSC (iPSC).

In some embodiments, the PSC comprises 1-20, optionally 8-10, copies of the engineered polynucleotide comprising the open reading frame encoding the protein selected from DLX5, HHEX, and FIGLA.

Yet other aspects of the present disclosure provide a composition comprising: a population of the PSC of any one of the preceding paragraphs or described elsewhere herein.

In some embodiments, the population comprises at least 2500/cm² of the PSC.

Some aspects of the present disclosure provide a method, comprising: culturing, in culture media, a population of pluripotent stem cells (PSCs) to produce an expanded population of PSCs; and expressing in PSCs of the expanded population a protein selected from DLX5, HHEX, and FIGLA to produce PGCLCs.

In some embodiments, the PSCs of the expanded population comprise an engineered polynucleotide comprising an open reading frame encoding DLX5.

In some embodiments, the PSCs of the expanded population comprise an engineered polynucleotide comprising an open reading frame encoding HHEX.

In some embodiments, the PSCs of the expanded population comprise an engineered polynucleotide comprising an open reading frame encoding FIGLA.

In some embodiments, the open reading frame of the engineered polynucleotide is operably linked to a heterologous promoter.

In some embodiments, the heterologous promoter is an inducible promoter.

In some embodiments, the population comprises 1×10²-1×10⁷ PSCs.

In some embodiments, the population of PSCs is cultured for about 3-5 days. In some embodiments, the population of PSCs is cultured for about 4 days.

In some embodiments, the PGCLCs are NANOS3⁺, SOX17⁺, TFAP2C⁺, PRDM1⁺, OCT4⁺, CD38⁺, EPCAM⁺, ITGA6⁺, and/or SOX2⁻ PGCLCs.

Some aspects of the present disclosure provide a method comprising:
(a) delivering to pluripotent stem cells (PSCs) an engineered polynucleotide comprising an inducible promoter operably linked to an open reading frame encoding a protein selected from DLX5, HHEX, and FIGLA;
(b) culturing the PSCs in feeder-free, serum-free culture media to produce an expanded population of PSCs; and
(c) culturing PSCs of the expanded population in a series of induction media comprising an inducing agent to produce NANOS3⁺, SOX17⁺, TFAP2C⁺, PRDM1⁺, OCT4⁺, CD38⁺, EPCAM⁺, ITGA6⁺, and/or SOX2⁻ PGCLCs.

In some embodiments, the engineered polynucleotide is a transposon and the delivering further comprises delivering a transposase to the PSCs.

In some embodiments, the inducible promoter is a chemically-inducible promoter, optionally a doxycycline-inducible promoter.

In some embodiments, the feeder-free, serum-free culture media of (b) comprises a solubilized basement membrane preparation extracted from the Engelbreth-Holm-Swarm (EHS) mouse sarcoma.

In some embodiments, the solubilized basement membrane preparation comprises extracellular matrix (ECM) proteins and growth factors.

In some embodiments, the ECM proteins are selected from Laminin, Collagen IV, heparan sulfate proteoglycans, and entactin/nidogen.

In some embodiments, the feeder-free, serum-free culture media of (b) comprises growth factors selected from recombinant human basic fibroblast growth factor (rh bFGF) and recombinant human transforming growth factor β (rh TGFβ).

In some embodiments, the culturing of (b) is for about 6 to about 24 hours.

In some embodiments, the PSCs of the expanded population of (c) are cultured at a density of about 2,000 cells/cm² to about 3,000 cells/cm².

In some embodiments, the culturing of (c) comprises culturing the PSCs is a first induction media, culturing the PSCs in a second induction media, culturing the PSCs in a third induction media, and culturing the PSCs in a fourth induction media.

In some embodiments, the first induction media comprises one or more of B-27, L-alanyl-L-glutamine, an inducing agent (e.g., doxycycline), Activin A, a glycogen synthase kinase (GSK) 3 inhibitor, and a selective FGFR1 and FGFR3 inhibitor.

In some embodiments, the second induction media comprises one or more of B-27, an inducing agent (e.g., doxycycline), a small molecule inhibitor of tankyrase (TNKS), and a human bone morphogenic protein 4 (hBMP4).

In some embodiments, the third induction media comprises one or more of B-27, an inducing agent (e.g., doxycycline), a small molecule inhibitor of tankyrase, stem cell factor (SCF), and epidermal growth factor (EGF).

In some embodiments, the fourth induction media comprises one or more of B-27, an inducing agent (e.g., doxycycline), a small molecule inhibitor of tankyrase, hBMP4, SCF, and EGF.

Other aspects of the present disclosure provide a PGCLC produced by the method of any one of the preceding claims.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B show identification of overexpressed transcription factors (TFs) that drive enhancement of NANOS3+ primordial germ cell yield. FIG. 1A shows the TFs were integrated into individual lines of NANOS3-mVenus PSCs and overexpressed via doxycycline induction during monolayer primordial germ cell formation. hPGCLC yield was compared in plus dox versus minus dox as well as compared to a no TF control condition.

FIG. 1B shows the TFs DLX5, HHEX and FIGLA enhanced NANOS3+ hPGCLC yield via the same protocol as discussed in FIG. 1A.

FIGS. 2A-2B show transcriptomic and proteomic analysis demonstrating that TFs drive on-target primordial germ cell formation. FIG. 2A shows transcriptomic characterization of hPGCLC. The results show upregulation of key hPGCLC genes and down regulation of PSCs, consistent with known positive controls. NANOS3+ hPGCLCs were isolated via FACS following TF-based or control induction and subjected to RNA-Sequencing. FIG. 2B shows analysis of key hallmarks of hPGCLC protein expression. The immunofluorescence was performed on TF-induced hPGCLCs for integrin (ITGA6), OCT4, and SOX17 expression.

FIGS. 3A-3C show the characterization of TF dynamics through dosage, time series and cytokine withdrawal. FIG. 3A shows the hPGCLC yield assessed following TF induction in the control condition, with no cytokines, and without hBMP4. Change in yield compared to normal condition is plotted, showing DLX5 retains 40% of its activity without hBMP4.

FIG. 3B shows the hPGCLC yield assessed under doxycycline dilution, showing increased doxycycline generally increases hPGCLC yield following TF induction. FIG. 3C shows the hPGCLC yield assessed after addition of doxycycline at various time points, showing TFs are generally beneficial when expressed throughout the differentiation process.

FIG. 4 shows a schematic of the TF-assisted hPGCLC formation method.

FIG. 5 shows TFs induce an increase in hPGCLC yield across different markers.

FIG. 6 shows TFs induce increase in hPGCLC yield across differentiation platforms.

FIG. 7 shows TF combination testing for hPGCLC formation.

### DETAILED DESCRIPTION

Primordial germ cells (PGCs) are the origin of gametogenesis, serving as the ancestral cell type for both oocyte and spermatocyte development (McLaren et al. 2003). Recently, a large number of techniques have been developed to differentiate human primordial germ cell-like cells (hPGCLCs) from human induced pluripotent stem cells (PSCs) (Mitsunage et al. 2017). Recently, new methods for monolayer hPGCLC differentiation have been developed that induce hPGCLC formation without embryoid bodies, allowing for ease of use and scalability. While many methods for hPGCLC induction exist, all suffer from large heterogeneity in hPGCLC yield depending on the cell line utilized, with many cell lines being defunct for germ cell formation. As primordial germ cells are utilized as the input cell type for in vitro ovarian and testis reconstitution, methods for high yield primordial germ cell formation are needed. Aspects of the present disclosure relate to a method of using direct transcription factor overexpression to induce differentiation of stem cells into NANOS3+ (Nanos C2HC-Type Zinc Finger 3), SOX17+ (SRY-Box Transcription Factor 17), TFAP2C+ (Transcription Factor AP-2 Gamma), PRDM1+ (PR/SET Domain 1), OCT4+ (Octamer Binding Transcription Factor 4), and/or SOX2− (SRY-Box Transcription Factor 2) primordial germ cells. It should be understood that the term “PGCLC” encompasses cells that express primordial germ cell-specific markers, such as NANOS3, SOX17, TFAP2C, PRDM1, OCT4, CD38, EPCAM, and/or ITGA6, and/or do not express SOX2, and exhibit other characteristics of naturally-occurring primordial germ cells.

### Primordial Germ Cell-Like Cells

Some aspects of the present disclosure provide primordial germ cell-like cells (PGCLCs) and methods of producing such cells. Primordial germ cells (PGCs) are the embryonic precursors of gametes (sperm and eggs), which generate a new organism that is capable of creating endless new generations through germ cells. PGCs represent the founder cells of the germline. PGCs are specified during early mammalian postimplantation development, and are uniquely programmed for transmission of genetic and epigenetic information to subsequent generations. Primordial germ cells are single cells that under certain culture conditions can form colonies of cells which morphologically resemble undifferentiated embryonic stem cells.

Primordial germ cells and PGCLCs express a number of different biomarkers that can be used to distinguish PGCLCs from other cell types. For example, PGCLCs cells are typically positive for Nanos C2HC-Type Zinc Finger 3 (NANOS3), SRY-Box Transcription Factor 17 (SOX17), Transcription Factor AP-2 Gamma (TFAP2C), PR/SET Domain 1 (PRDM1), Octamer Binding Transcription Factor 4 (OCT4), and/or negative for SRY-Box Transcription Factor 2 (SOX2).

There are other characteristics of PGCLCs that distinguish them from non-PGCLCs including, but not limited to, their global decrease in 5-methyl-cytosine levels and H3K9me2 levels compared to stem cells as well as their CXCL12/SDF1-guided chemotaxis movement, and cytoplasmic granules.

### Pluripotent Stem Cells

The PGCLCs provided herein are differentiated from pluripotent stem cells. Pluripotent stem cells are cells that have the capacity to self-renew by dividing, and to develop into the three primary germ cell layers of the early embryo (e.g., ectoderm, endoderm, and mesoderm), and therefore into all cells of the adult body, but not extra-embryonic tissues such as the placenta (Shi et al. 2017).

Non-limiting examples of pluripotent stem cells include induced pluripotent cell (iPSCs), “true” embryonic stem cell (ESCs) derived from embryos, embryonic stem cells made by somatic cell nuclear transfer (ntESCs), and embryonic stem cells from unfertilized eggs (parthenogenesis embryonic stem cells, or pESCs). In some embodiments, a pluripotent cell is a human pluripotent cell.

In some embodiments, a pluripotent stem cell is an embryonic stem cell, such as a human embryonic stem cell. “Embryonic stem cell” is a general term for pluripotent stem cells that are made using embryos or eggs, rather than for cells genetically reprogrammed from the body. As used herein, “ESCs” encompass true ESCs, ntESCs, and pESCs.

In other embodiments, a pluripotent stem cell is an induced pluripotent stem cell, such as a human induced pluripotent stem cell. iPSCs may be derived from skin or blood cells that have been reprogrammed back into an embryonic-like pluripotent state that enables the development of an unlimited source of any type of human cell.

Some aspects of the present disclosure provide a PSC comprising: a protein selected from DLX5, HHEX, and FIGLA, wherein the protein is expressed or overexpressed. In some embodiments, the protein is expressed at a level that is at least 5%, at least 10%, at least 15%, at least 20%, at least 25%, at least 50%, or at least 100% higher than a control level. In some embodiments, a control level is an endogenous level of the protein, for example in a naturally-occurring pluripotent stem cell. In some embodiments, a PSC comprises DLX5. In some embodiments, a PSC expresses or overexpresses DLX5. In some embodiments, a PSC comprises HHEX. In some embodiments, a PSC expresses or overexpresses HHEX. In some embodiments, a PSC comprises FIGLA. In some embodiments, a PSC expresses or overexpresses FIGLA.

Data provided herein shows that expression of only one of DLX5, HHEX, or FIGLA outperforms combinatorial expression of all three. For example, combinatorial expression of DLX5, HHEX, and FIGLA in PSCs results in a 2-fold increase in efficiency of NANOS3⁺, SOX17⁺, TFAP2C⁺, PRDM1V, OCT4⁺, CD38⁺, EPCAM⁺, ITGA6⁺, and/or SOX2⁻ PGCLC production, relative to a no TF control. Unexpectedly, however, combinatorial expression of DLX5, HHEX, and FIGLA in PSCs results in a 5 to 45 fold decrease in efficiency, relative to a PSC control expressing only one of DLX5, HHEX, or FIGLA. Thus, in some embodiments, a PSC comprises DLX5, HHEX, or FIGLA, but not all three together.

In other embodiments, a PSC comprises DLX5 and HHEX. In some embodiments, a PSC expresses or overexpresses DLX5 and HHEX. In some embodiments, a PSC comprises DLX5 and FIGLA. In some embodiments, a PSC expresses or overexpresses DLX5 and FIGLA. In some embodiments, a PSC comprises HHEX and FIGLA. In some embodiments, a PSC expresses or overexpresses HHEX and FIGLA.

### Transcription Factors

The PGCLCs provided herein are differentiated from pluripotent stem cells, in some embodiments, by expressing one or more (e.g., 2, 3, 4, 5, 6, 7, 8, or 9) transcription factors (i.e., a protein that controls the rate of transcription). Differentiation is the process by which an uncommitted cell or a partially committed cell commits to a specialized cell fate. Aspects of the present disclosure relate to the differentiation of uncommitted pluripotent stem cells into a PGCLC fate.

In some embodiments, the transcription factors are selected from DLX5, HHEX, and FIGLA. In some embodiments, pluripotent stem cells, such as hPSCs or hiPSCs, are engineered to express or overexpress DLX5. In some embodiments, pluripotent stem cells, such as hPSCs or hiPSCs, are engineered to express or overexpress HHEX. In some embodiments, pluripotent stem cells, such as hPSCs or hiPSCs, are engineered to express or overexpress FIGLA. In some embodiments, pluripotent stem cells, such as hPSCs or hiPSCs, are engineered to express or overexpress DLX5 and HHEX. In some embodiments, pluripotent stem cells, such as hPSCs or hiPSCs, are engineered to express or overexpress DLX5 and FIGLA. In some embodiments, pluripotent stem cells, such as hPSCs or hiPSCs, are engineered to express or overexpress HHEX and FIGLA. In some embodiments, pluripotent stem cells, such as hPSCs or hiPSCs, are engineered to express or overexpress DLX5, HHEX, and FIGLA.

A cell “expressed” a particular protein if the level of the protein in the cell is detectable (e.g., using a known protein assay). A cell “overexpresses” a particular protein (e.g., engineered polynucleotide encoding the protein) if the level of the protein is higher than (e.g., at least 5%, at least 10%, or at least 20% higher than) the level of the protein expressed from an endogenous, naturally-occurring polynucleotide encoding the protein.

### Engineered Polynucleotides and Polypeptides

The pluripotent stem cells of the present disclosure, in some embodiments, comprise engineered polynucleotides. An engineered polynucleotide is a nucleic acid (e.g., at least two nucleotides covalently linked together, and in some instances, containing phosphodiester bonds, referred to as a phosphodiester backbone) that does not occur in nature. Engineered polynucleotides include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered polynucleotide may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a polynucleotide is a complementary DNA (cDNA). cDNA is synthesized from a single-stranded RNA (e.g., messenger RNA (mRNA) or microRNA (miRNA)) template in a reaction catalyzed by reverse transcriptase.

Engineered polynucleotides of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered polynucleotides may be used in accordance with the present disclosure.

In some embodiments, an engineered polynucleotide comprises a promoter operably linked to an open reading frame. A promoter is a nucleotide sequence to which RNA polymerase binds to initial transcription (e.g., ATG). Promoters are typically located directly upstream from (at the 5′ end of) a transcription initiation site. In some embodiments, a promoter is a heterologous promoter. A heterologous promoter is not naturally associated with the open reading frame to which is it operably linked.

In some embodiments, a promoter is an inducible promoter. An inducible promoter may be regulated in vivo by a chemical agent, temperature, or light, for example. Inducible promoters enable, for example, temporal and/or spatial control of gene expression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells). In some embodiments, the inducible promoter is a tetracycline-inducible promoter. In some embodiments, the inducible promoter is a doxycycline-inducible promoter. In other embodiments, a promoter is a constitutive promoter (active in vivo, unregulated).

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

Vectors used for delivery of an engineered polynucleotide include minicircles, plasmids, bacterial artificial chromosomes (BACs), and yeast artificial chromosomes. Transposon-based systems, such as the piggyBac™ system (e.g., Chen et al. Nature Communications. 2020; 11(1): 3446), is also contemplated herein.

A pluripotent stem cells, in some embodiments, comprises an engineered polynucleotide comprising an open reading frame encoding a protein selected from DLX5, HHEX, and FIGLA. In some embodiments, the engineered polynucleotide comprises an open reading frame encoding DLX5. In some embodiments, the engineered polynucleotide comprises an open reading frame encoding HHEX. In some embodiments, the engineered polynucleotide comprises an open reading frame encoding FIGLA.

In some embodiments, a pluripotent stem cell comprises an engineered polynucleotide comprising an open reading frame encoding DLX5 and an engineered polynucleotide comprising an open reading frame encoding HHEX. In some embodiments, a pluripotent stem cell comprises an engineered polynucleotide comprising an open reading frame encoding DLX5 and an engineered polynucleotide comprising an open reading frame encoding FIGLA. In some embodiments, a pluripotent stem cell comprises an engineered polynucleotide comprising an open reading frame encoding HHEX and an engineered polynucleotide comprising an open reading frame encoding FIGLA. In some embodiments, a pluripotent stem cell comprises an engineered polynucleotide comprising an open reading frame encoding DLX5, an engineered polynucleotide comprising an open reading frame encoding HHEX, and an engineered polynucleotide comprising an open reading frame encoding FIGLA.

An engineered polynucleotide encoding comprising an open reading frame encoding Folliculogenesis Specific BHLH Transcription Factor (FIGLA) (e.g., UniprotKB Accession No. Q6QHK4), in some embodiments, encodes a protein comprising the sequence of:

(SEQ ID NO: 1)
MDPAPGVLDPRAAPPALLGTPQAEVLEDVLREQFGPLPQLAAVCR
LKRLPSGGYSSTENLQLVLERRRVANAKERERIKNLNRGFARLKA
LVPFLPQSRKPSKVDILKGATEYIQVLSDLLEGAKDSKKQDPDEQ
SYSNNSSESHISSARQLSRNITQHISCAFGLKNEEEGPWADGGSG
EPAHACRHSVMSTTEIISPTRSLDRFPEVELLSHRLPQV

An engineered polynucleotide encoding comprising an open reading frame encoding Distal-Less Homeobox 5 (DLX5) (e.g., UniprotKB Accession No. P56178), in some embodiments, encodes a protein comprising the sequence of:

(SEQ ID NO: 2)
MTGVFDRRVPSIRSGDFQAPFQTSAAMHHPSQESPTLPESSATDS
DYYSPTGGAPHGYCSPTSASYGKALNPYQYQYHGVNGSAGSYPAK
AYADYSYASSYHQYGGAYNRVPSATNQPEKEVTEPEVRMVNGKPK
KVRKPRTIYSSFQLAALQRRFQKTQYLALPERAELAASLGLTQTQ
VKIWFQNKRSKIKKIMKNGEMPPEHSPSSSDPMACNSPQSPAVWE
PQGSSRSLSHHPHAHPPTSNQSPASSYLENSASWYTSAASSINSH
LPPPGSLQHPLALASGTLY

An engineered polynucleotide encoding comprising an open reading frame encoding Hematopoietically Expressed Homeobox (HHEX) (e.g., UniprotKB Accession No. Q03014), in some embodiments, encodes a protein comprising the sequence of:

(SEQ ID NO: 3)
MQYPHPGPAAGAVGVPLYAPTPLLQPAHPTPFYIEDILGRGPAAP
TPAPTLPSPNSSFTSLVSPYRTPVYEPTPIHPAFSHHSAAALAAA
YGPGGFGGPLYPFPRTVNDYTHALLRHDPLGKPLLWSPFLQRPLH
KRKGGQVRFSNDQTIELEKKFETQKYLSPPERKRLAKMLQLSERQ
VKTWFQNRRAKWRRLKQENPQSNKKEELESLDSSCDQRQDLPSEQ
NKGASLDSSQCSPSPASQEDLESEISEDSDQEVDIEGDKSYFNAG

The number of copies of an engineered polynucleotide delivered to a PSC may vary. In some embodiments, a PSC comprises 1-20 copies of an engineered polynucleotide. For example, and PSC may comprise 1-15, 1-10, 2-10, 2-15, 2-10, 5-20, 5-15, or 5-10 copies of an engineered polynucleotide. In some embodiments, a PSC comprises 8-10 copies of an engineered polynucleotide. Greater than 20 copies are also contemplated herein.

### Methods of Producing PGCLCs

The methods of producing PGCLCs provided herein, in some aspects, comprises culturing, in culture media, a population of pluripotent stem cells (PSCs) to produce an expanded population of PSCs; and expressing in PSCs of the expanded population a protein selected from DLX5, HHEX, and FIGLA to produce PGCLCs.

In some embodiments, the PSCs of the expanded population comprise an engineered polynucleotide comprising an open reading frame encoding DLX5. In some embodiments, the PSCs of the expanded population comprise an engineered polynucleotide comprising an open reading frame encoding HHEX. In some embodiments, the PSCs of the expanded population comprise an engineered polynucleotide comprising an open reading frame encoding FIGLA.

In some embodiments, the open reading frame of the engineered polynucleotide is operably linked to a heterologous promoter.

In some embodiments, the heterologous promoter is an inducible promoter, non-limiting examples of which are provided elsewhere herein.

The population a starting population comprises about 1×10²-1×10¹⁰, about 1×10²-1×10⁹, about 1×10²-1×10⁸, or about 1×10²-1×10⁷ PSCs. In some embodiments, the population comprises about 1×10³-1×10⁸ or about 1×10³-1×10⁷ PSCs. In some embodiments, the population comprises about 1×10⁴-1×10⁷ or about 1×10⁵-1×10⁶ PSCs. In some embodiments, the population comprises about 1×10¹ PSCs, about 1×10² PSCs, about 1×10³ PSCs, about 1×10⁴ PSCs, about 1×10⁵ PSCs, about 1×10⁶ PSCs, about 1×10⁷ PSCs, about 1×10⁸ PSCs, about 1×10⁹ PSCs, or about 1×10¹⁰ PSCs.

In some embodiments, the population of PSCs is cultured for about 2 to about 6 days, about 2 to about 5 days, about 2 to about 4 days, about 3 to about 6 days, about 3 to about 5 days, or about 3 to about 4 days. In some embodiments, the population of PSCs is cultured for about 2 days, about 3 days, about 4 days, about 5 days, or about 6 days.

Some methods of the present disclosure provide methods comprising (a) delivering to PSCs an engineered polynucleotide comprising an inducible promoter operably linked to an open reading frame encoding a protein selected from DLX5, HHEX, and FIGLA; (b) culturing the PSCs in feeder-free, serum-free culture media to produce an expanded population of PSCs; and (c) culturing PSCs of the expanded population in a series of induction media comprising an inducing agent to produce NANOS3⁺, SOX17⁺, TFAP2C⁺, PRDM1⁺, OCT4⁺, CD38⁺, EPCAM⁺, ITGA6⁺, and/or SOX2⁻ PGCLCs. In some embodiments, the series of induction media comprises a first, a second, a third, and a fourth induction media.

In some embodiments, the PSCs are cultured in feeder-free, serum-free culture media for about 6 to about 24 hours. For example, the PSC may be cultured in feeder-free, serum-free culture media for about, 6 to about 12 hours. In some embodiments, the PSCs are cultured in feeder-free, serum-free culture media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In some embodiments, the expanded population of PSCs comprises at least 5×10³ PSCs. For example, the expanded population (e.g., at the time of induction) may comprise at least 1×10⁴, at least 1×10⁵, at least 1×10⁶, or at least 1×10⁷ PSCs. In some embodiments, the expanded population of PSCs comprises about 5×10³ PSCs to about 1×10⁷ PSCs.

In some embodiments, PSCs of the expanded population are cultured at a density of about 2,000 cells/cm² to about 3,000 cells/cm². In some embodiments, PSCs of the expanded population are cultured at a density of about 500/cm²-10000/cm² PSCs. In some embodiments, the PSCs of the expanded population are cultured at a density of about 1000/cm²-9500/cm² PSCs. In some embodiments, PSCs of the expanded population are cultured at a density of about 1500/cm²-9000/cm² PSCs. In some embodiments, PSCs of the expanded population are cultured at a density of about 2000/cm²-8500/cm² PSCs. In some embodiments, PSCs of the expanded population are cultured at a density of about 2500/cm²-8000/cm² PSCs. In some embodiments, PSCs of the expanded population are cultured at a density of about 3000/cm²-7500/cm² PSCs. In some embodiments, PSCs of the expanded population are cultured at a density of about 3500/cm²-7000/cm² PSCs. In some embodiments, the population comprises 4000/cm²-6500/cm² PSCs. In some embodiments, PSCs of the expanded population are cultured at a density of about 4500/cm²-6000/cm² PSCs. In some embodiments, PSCs of the expanded population are cultured at a density of about 5000/cm²-5500/cm² PSCs. In some embodiments, PSCs of the expanded population are cultured at a density of at least 500/cm² PSCs, at least 1000/cm² PSCs, at least 1500/cm² PSCs, at least 2000/cm² PSCs, at least 2500/cm² PSCs, at least 3000/cm² PSCs, at least 3500/cm² PSCs, at least 4000/cm² PSCs, at least 4500/cm² PSCs, at least 5000/cm² PSCs, at least 5500/cm² PSCs, at least 6000/cm² PSCs, at least 6500/cm² PSCs, at least 7000/cm² PSCs, at least 7500/cm² PSCs, at least 8000/cm² PSCs, at least 8500/cm² PSCs, at least 9000/cm² PSCs, at least 9500/cm² PSCs, or at least 10000/cm² PSCs.

In some embodiments, PSCs of the expanded population are cultured for no longer than 8 days, no longer than 7 days, no longer than 6 days, no longer than 5 days, or no longer than 4 days. For example, PSCs of the expanded population may be cultured for about 2 to about 8 days, about 2 to about 7 days, about 2 to about 6 days, about 2 to about 5 days, about 2 to about 4 days, about 3 to about 8 days, about 3 to about 7 days, about 3 to about 6 days, about 3 to about 5 days, or about 3 to about 4 days. In some embodiments, PSCs of the expanded population are cultured for about 2 days, about 3 days, about 4 days, about 5 days, about 6 days, about 7 days, or about 8 days.

In some embodiments, PSCs of the expanded population are cultured in a first induction media for about 6 to about 36 hours. For example, the PSC may be cultured in a first induction media for about 6 to about 24 hours, about 6 to about 18 hours, about 6 to about 12 hours, 12 to about 36 hours, about 12 to about 24 hours, about 12 to about 18 hours, 18 to about 36 hours, or about 18 to about 24 hours. In some embodiments, the PSCs are cultured in a first induction media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours.

In some embodiments, PSCs of the expanded population are cultured in a second induction media for about 6 to about 36 hours. For example, the PSC may be cultured in a second induction media for about 6 to about 24 hours, about 6 to about 18 hours, about 6 to about 12 hours, 12 to about 36 hours, about 12 to about 24 hours, about 12 to about 18 hours, 18 to about 36 hours, or about 18 to about 24 hours. In some embodiments, the PSCs are cultured in a second induction media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours.

In some embodiments, PSCs of the expanded population are cultured in a third induction media for about 6 to about 36 hours. For example, the PSC may be cultured in a third induction media for about 6 to about 24 hours, about 6 to about 18 hours, about 6 to about 12 hours, 12 to about 36 hours, about 12 to about 24 hours, about 12 to about 18 hours, 18 to about 36 hours, or about 18 to about 24 hours. In some embodiments, the PSCs are cultured in a third induction media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours.

In some embodiments, PSCs of the expanded population are cultured in a fourth induction media for about 6 to about 36 hours. For example, the PSC may be cultured in a fourth induction media for about 6 to about 24 hours, about 6 to about 18 hours, about 6 to about 12 hours, 12 to about 36 hours, about 12 to about 24 hours, about 12 to about 18 hours, 18 to about 36 hours, or about 18 to about 24 hours. In some embodiments, the PSCs are cultured in a fourth induction media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours.

### Transfection Methods

The engineered polynucleotide of the present disclosure may be delivered to a PSC using any one or more transfection method, including chemical transfection methods, viral transduction methods, and electroporation.

In some embodiments, an engineered polynucleotide is delivered on a vector. A vector is any vehicle, for example, a virus or a plasmid, that is used to transfer a desired polynucleotide into a host cell, such as a PSC. In some embodiments, the vector is a viral vector. In some embodiments, a viral vector is not a naturally occurring viral vector. The viral vector may be from adeno-associated virus (AAV), adenovirus, herpes simplex virus, lentiviral, retrovirus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B 19. Other viral vectors are encompassed by the present disclosure.

In some embodiments, a viral vector is an AAV vector. AAV is a small, non-enveloped virus that packages a single-stranded linear DNA genome that is approximately 5 kb long and has been adapted for use as a gene transfer vehicle (Samulski, R J et al., Annu Rev Virol. 2014; 1(1):427-51). The coding regions of AAV are flanked by inverted terminal repeats (ITRs), which act as the origins for DNA replication and serve as the primary packaging signal (McLaughlin, S K et al. Virol. 1988; 62(6): 1963-73; Hauswirth, W W et al. 1977; 78(2):488-99). Thus, an AAV vector typically includes ITR sequences. Both positive and negative strands are packaged into virions equally well and capable of infection (Zhong, L et al. Mol Ther. 2008; 16(2):290-5; Zhou, X et al. Mol Ther. 2008; 16(3):494-9; Samulski, R J et al. Virol. 1987; 61(10):3096-101). In addition, a small deletion in one of the two ITRs allows packaging of self-complementary vectors, in which the genome self-anneals after viral uncoating. This results in more efficient transduction of cells but reduces the coding capacity by half (McCarty, D M et al. Mol Ther. 2008; 16(10): 1648-56; McCarty, D M et al. Gene Ther. 2001; 8(16): 1248-54).

In some embodiments, a polynucleotide is delivered to a cell using a transposon/transposase system. For example, the piggyBac™ transposon system may be used. A piggyBac™ transposon is a mobile genetic element that efficiently transposes between vectors and chromosomes via a “cut and paste” mechanism (Woodard et al. 2015). During transposition, the piggyBac™ transposase recognizes transposon-specific inverted terminal repeat sequences (ITRs) located on both ends of the transposon vector and efficiently moves the contents from the original sites and integrates them into TTAA chromosomal sites. The piggyBac™ transposon system facilitates efficient integration of a polynucleotide into a cell genome.

Thus, in some embodiments, the method further comprises delivering to a PSC a transposon comprising an engineered polynucleotide and also delivering a transposase.

In some embodiments, an engineered polynucleotide is delivered to a cell using electroporation. Electroporation is a physical transfection method that uses an electrical pulse to create temporary pores in cell membranes through which the engineered polynucleotide can pass into cells. See, e.g., Chicaybam L et al. Front. Bioeng. Biotechnol., 23 Jan. 2017.

Following transfection, the engineered polynucleotides may be integrated into the genome of a PSC. In some embodiments, an engineered polynucleotide may further comprise an antibiotic resistance gene to confer resistance to an antibiotic used in an antibiotic drug selection process. In this way, a ‘pure’ population of cells comprising an integrated engineered polynucleotide may be obtained. In some embodiments, a population of cells comprising an integrated engineered polynucleotide are selected using antibiotic drug selection. Antibiotic drug selection is the process of treating a population of cells with an antibiotic so that only cells that are capable of surviving in the presence of said antibiotic will remain in the population. Non-limiting examples of antibiotics that may be used for antibiotic drug selection include: puromycin, blasticidin, geneticin, hygromycin, mycophenolic acid, zeocin, carbenicillin, kanemycin, ampicillin, and actinomycin.

### Culture Media

The methods provided herein, in some embodiments, comprise culturing PSCs in a feeder-free, serum-free culture media. Culture media may comprise, for example, a solubilized basement membrane preparation extracted from the Engelbreth-Holm-Swarm (EHS) mouse sarcoma (e.g., Corning® Matrigel® Matrix) (coated at 75 to 150 μl per cm² of lot-based diluted suspension). In some embodiments, the solubilized basement membrane preparation comprises one or more extracellular matrix (ECM) protein and one or more growth factor. For example, the ECM proteins may be selected from Laminin, Collagen IV, heparan sulfate proteoglycans, and entactin/nidogen.

In some embodiments, culture media further comprises one or more growth factor, for example, selected from recombinant human basic fibroblast growth factor (rh bFGF) (e.g., 80 ng/ml to 120 ng/ml) and recombinant human transforming growth factor β (rh TGFβ) (e.g., 20 to 25 pM). In some embodiments, culture media further comprises rh bFGF and rh TGFβ. In some embodiments, culture media comprises mTeSR™ media (STEMCELL Technologies).

In some embodiments, a first induction media comprises one or more of (e.g., 2, 3, 4, or more of) B-27 Supplement (e.g., 90× to 110×), L-alanyl-L-glutamine (e.g., 1.8 mM to 2.2 mM), an inducing agent (e.g., doxycycline (e.g., 50 ng/ml to 2000 ng/ml)), Activin A (e.g., 50 ng/ml to 150 ng/ml), a glycogen synthase kinase (GSK) 3 inhibitor (e.g., 2.8 μM to 3.2 μM), a selective FGFR1 and FGFR3 inhibitor (e.g., 90 nM to 110 nM), and a small molecule ROCK inhibitor (e.g., 8 μM to 12 μM). In some embodiments, a first induction media comprises B-27, L-alanyl-L-glutamine, an inducing agent (e.g., doxycycline), Activin A, a glycogen synthase kinase (GSK) 3 inhibitor, and a selective FGFR1 and FGFR3 inhibitor. For example, the first induction media may comprise aRB27 Media, doxycycline, Activin A, CHIR99021, and PD 173074.

In some embodiments, the second induction media comprises one or more of (e.g., 2, 3, 4, or more of) B-27 Supplement (e.g., 90× to 110×), an inducing agent (e.g., doxycycline (e.g., 50 ng/ml to 2000 ng/ml)), a small molecule inhibitor of tankyrase (TNKS) (e.g., 0.9 μM to 1.1 μM), and a human bone morphogenic protein 4 (hBMP4) (e.g., 20 ng/ml to 250 ng/ml). In some embodiments, the second induction media comprises B-27, an inducing agent (e.g., doxycycline), a small molecule inhibitor of tankyrase (TNKS), and a human bone morphogenic protein 4 (hBMP4). For example, the second induction media may comprise aRB27 Media, doxycycline, XAV939, and human bone morphogenic protein 4 (hBMP4).

In some embodiments, the third induction media comprises one or more of (e.g., 2, 3, 4, or more of) B-27, an inducing agent (e.g., doxycycline), a small molecule inhibitor of tankyrase (e.g., 0.9 μM to 1.1 μM), stem cell factor (SCF) (e.g., 25 ng/ml to 200 ng/ml), and epidermal growth factor (EGF) (e.g., 25 ng/ml to 100 ng/ml). In some embodiments, the third induction media comprises B-27 Supplement (e.g., 90× to 110×), an inducing agent (e.g., doxycycline (e.g., 50 ng/ml to 2000 ng/ml)), a small molecule inhibitor of tankyrase (e.g., 0.9 μM to 1.1 μM), stem cell factor (SCF) (e.g., 25 ng/ml to 200 ng/ml), and epidermal growth factor (EGF) (e.g., 25 ng/ml to 100 ng/ml). For example, the third induction media may comprise aRB27 Media, doxycycline, XAV939, SCF, and EGF.

In some embodiments, the fourth induction media comprises one or more of (e.g., 2, 3, 4, or more of) B-27 Supplement (90-110×), an inducing agent (e.g., doxycycline (e.g., 50 ng/ml to 2000 ng/ml)), a small molecule inhibitor of tankyrase (e.g., 0.9 μM to 1.1 μM), hBMP4 (e.g., 20 ng/ml to 250 ng/ml), SCF (e.g., 25 ng/ml to 200 ng/ml), and EGF (e.g., 25 ng/ml to 100 ng/ml). In some embodiments, the fourth induction media comprises B-27, an inducing agent (e.g., doxycycline), a small molecule inhibitor of tankyrase, hBMP4, SCF, and EGF. For example, the fourth induction media may comprise aRB27 Media, doxycycline, XAV939, hBMP4, SCF, and EGF.

The ‘aRB27 Media’ used herein comprises Advanced RPMI, B-27™ Supplement, minus vitamin A (Thermo Fisher) or plus vitamin A, GlutaMAX™ Supplement (Thermo Fisher), non-essential amino acids (NEAA), Primocin® (a broad-spectrum antibiotic), and Y-27632 (a small molecule ROCK inhibitor).

GlutaMAX™ Supplement comprises L-alanyl-L-glutamine, which is a dipeptide substitute for L-glutamine.

Activin-A is a dimeric glycoprotein, which belongs to the transforming growth factor-β (TGF-β) family.

CHIR99021 is an aminopyrimidine derivative that is an extremely potent glycogen synthase kinase (GSK) 3 inhibitor, inhibiting both GSK3β (IC₅₀=6.7 nM) and GSK3α (IC₅₀=10 nM). GSK3 is a serine/threonine kinase that is a key inhibitor of the WNT pathway; therefore, CHIR99021 functions as a WNT activator.

PD 173074 is a selective FGFR1 and FGFR3 inhibitor (IC50 values are ˜5 nM, ˜21.5 nM, ˜100 nM, ˜17600 nM and ˜19800 nM for FGFR3, FGFR1, VEGFR2, PDGFR and c-Src respectively, and >50000 nM for EGFR, InsR, MEK and PKC).

XAV939 is a potent, small molecule inhibitor of tankyrase (TNKS) 1 and 2 (IC₅₀=11 and 4 nM, respectively) (Huang et al.). By inhibiting TNKS activity, XAV939 increases the protein levels of the axin-GSK3β complex and promotes the degradation of β-catenin in SW480 cells (Huang et al.), thereby inhibiting WNT pathway downstream actions.

### Therapeutic Compositions and Method of Use

The present disclosure provides, in some embodiments, therapeutic compositions comprising the PGCLCs produced herein. In some embodiments, the compositions further comprise a pharmaceutically-acceptable excipient. The compositions, in some embodiments, are cryopreserved.

Such compositions may be administered to a subject, such as a human subject, using any suitable route of administration. Suitable routes of administration include, for example, parenteral routes such as intravenous, intrathecal, parenchymal, or intraventricular routes. Suitable routes of administration include, for example, parenteral routes such as intravenous, intrathecal, parenchymal, or intraventricular injection.

In some embodiments, a subject is a human subject Subjects that could benefit from such composition include patients struggling with male or female factor infertility, in which no viable gametes can be produced.

The compositions may be administered to a subject in a therapeutically effective amount. The term “therapeutically effective amount” refers to the amount of PGCLCs required to confer therapeutic effect on a subject, either alone or in combination with at least one other active agent. Effective amounts vary, as recognized by those skilled in the art, depending on the route of administration, excipient usage, and co-usage with other active agents. The quantity to be administered depends on the subject to be treated, including, for example, the strength of an individual's immune system or genetic predispositions. Suitable dosage ranges are readily determinable by one skilled in the art and may be on the order of micrograms of the polypeptide of this disclosure. The dosage of the preparations disclosed herein may depend on the route of administration and varies according to the size of the subject.

It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited in the present application are incorporated by reference for the purposes or subject matter referenced in this disclosure.

### ADDITIONAL EMBODIMENTS

The disclosure also relates to the additional embodiments set out in the numbered paragraphs below:

1. A pluripotent stem cell (PSC) comprising: an engineered polynucleotide comprising an open reading frame encoding a protein selected from DLX5, HHEX, and FIGLA.

2. The PSC of paragraph 1, wherein the PSC comprises the engineered polynucleotide comprising an open reading frame encoding DLX5.

3. The PSC of paragraph 1 or 2, wherein the PSC comprises the engineered polynucleotide comprising an open reading frame encoding HHEX.

4. The PSC of any one of the preceding paragraphs, wherein the PSC comprises the engineered polynucleotide comprising an open reading frame encoding FIGLA.

5. The PSC of any one of the preceding paragraphs, wherein the PSC expresses or overexpresses: DLX5; HHEX; FIGLA; DLX5 and HHEX; DLX5 and FIGLA; HHEX and FIGLA; or DLX5, HHEX, and FIGLA.

6. The PSC of any one of the preceding paragraphs, wherein the open reading frame of the engineered polynucleotide is operably linked to a heterologous promoter.

7. The PSC of paragraph 6, wherein the heterologous promoter is an inducible promoter.

8. A pluripotent stem cell (PSC) comprising: a protein selected from DLX5, HHEX, and FIGLA, wherein the protein is overexpressed.

9. The PSC of paragraph 8, wherein the PSC expresses or overexpresses: DLX5; HHEX; FIGLA; DLX5 and HHEX; DLX5 and FIGLA; HHEX and FIGLA; or DLX5, HHEX, and FIGLA.

10. The PSC of any one of the preceding paragraphs, wherein the PSC is a human PSC.

11. The PSC of any one of the preceding paragraphs, wherein the PSC is an induced PSC (iPSC).

12. The PSC of any one of the preceding paragraphs, wherein the PSC comprises 1-20, optionally 8-10, copies of the engineered polynucleotide comprising the open reading frame encoding the protein selected from DLX5, HHEX, and FIGLA.

13. A composition comprising: a population of the PSC of any one of the preceding paragraphs or described elsewhere herein.

14. The composition of paragraph 13, wherein the population comprises at least 2500/cm² of the PSC.

15. A method, comprising: culturing, in culture media, a population of pluripotent stem cells (PSCs) to produce an expanded population of PSCs; and expressing in PSCs of the expanded population a protein selected from DLX5, HHEX, and FIGLA to produce PGCLCs.

16. The method of paragraph 15, wherein the PSCs of the expanded population comprise an engineered polynucleotide comprising an open reading frame encoding DLX5.

17. The method of paragraph 15 or 16, wherein the PSCs of the expanded population comprise an engineered polynucleotide comprising an open reading frame encoding HHEX.

18. The method of any one of paragraphs 15-17, wherein the PSCs of the expanded population comprise an engineered polynucleotide comprising an open reading frame encoding FIGLA.

19. The method of any one of the preceding paragraphs, wherein the open reading frame of the engineered polynucleotide is operably linked to a heterologous promoter.

20. The method of any one of the preceding paragraphs, wherein the heterologous promoter is an inducible promoter.

21. The method of any one of the preceding paragraphs, wherein the population comprises 1×10²-1×10⁷ PSCs.

22. The method of any one of the preceding paragraphs, wherein the population of PSCs is cultured for about 3-5 days.

23. The method of paragraph 22, wherein the population of PSCs is cultured for about 4 days.

24. The method of any one of the preceding paragraphs, wherein the PGCLCs are NANOS3⁺, SOX17⁺, TFAP2C⁺, PRDM1⁺, OCT4⁺, CD38⁺, EPCAM⁺, ITGA6⁺, and/or SOX2⁻

### PGCLCs.

25. A method comprising:
(a) delivering to pluripotent stem cells (PSCs) an engineered polynucleotide comprising an inducible promoter operably linked to an open reading frame encoding a protein selected from DLX5, HHEX, and FIGLA;
(b) culturing the PSCs in feeder-free, serum-free culture media to produce an expanded population of PSCs; and
(c) culturing PSCs of the expanded population in a series of induction media comprising an inducing agent to produce NANOS3⁺, SOX17⁺, TFAP2C⁺, PRDM1⁺, OCT4⁺, CD38⁺, EPCAM⁺, ITGA6⁺, and/or SOX2⁻ PGCLCs.

26. The method of paragraph 25, wherein the engineered polynucleotide is a transposon and the delivering further comprises delivering a transposase to the PSCs.

27. The method of paragraph 25 or 26, wherein the inducible promoter is a chemically-inducible promoter, optionally a doxycycline-inducible promoter.

28. The method of any one of paragraphs 25-27, wherein the feeder-free, serum-free culture media of (b) comprises a solubilized basement membrane preparation extracted from the Engelbreth-Holm-Swarm (EHS) mouse sarcoma.

29. The method of paragraph 28, wherein the solubilized basement membrane preparation comprises extracellular matrix (ECM) proteins and growth factors.

30. The method of paragraph 29, wherein the ECM proteins are selected from Laminin, Collagen IV, heparan sulfate proteoglycans, and entactin/nidogen.

31. The method of any one of paragraphs 25-30, wherein the feeder-free, serum-free culture media of (b) comprises growth factors selected from recombinant human basic fibroblast growth factor (rh bFGF) and recombinant human transforming growth factor β (rh TGFβ).

32. The method of any one of paragraphs 25-31, wherein the culturing of (b) is for about 6 to about 24 hours.

33. The method of any one of paragraphs 25-32, wherein the PSCs of the expanded population of (c) are cultured at a density of about 2,000 cells/cm² to about 3,000 cells/cm².

34. The method of any one of paragraphs 25-33, wherein the culturing of (c) comprises culturing the PSCs is a first induction media, culturing the PSCs in a second induction media, culturing the PSCs in a third induction media, and culturing the PSCs in a fourth induction media.

35. The method of paragraph 34, wherein the first induction media comprises one or more of B-27, L-alanyl-L-glutamine, an inducing agent (e.g., doxycycline), Activin A, a glycogen synthase kinase (GSK) 3 inhibitor, and a selective FGFR1 and FGFR3 inhibitor.

36. The method of paragraph 34 or 35, wherein the second induction media comprises one or more of B-27, an inducing agent (e.g., doxycycline), a small molecule inhibitor of tankyrase (TNKS), and a human bone morphogenic protein 4 (hBMP4).

37. The method of any one of paragraphs 34-36, wherein the third induction media comprises one or more of B-27, an inducing agent (e.g., doxycycline), a small molecule inhibitor of tankyrase, stem cell factor (SCF), and epidermal growth factor (EGF).

38. The method of any one of paragraphs 34-37, wherein the fourth induction media comprises one or more of B-27, an inducing agent (e.g., doxycycline), a small molecule inhibitor of tankyrase, hBMP4, SCF, and EGF.

39. A primordial germ cell-like cell produced by the method of any one of the preceding paragraphs.

### EXAMPLES

### Example 1. Production of primordial germ cells

Primordial germ cells (PGCs) are the origin of gametogenesis, serving as the ancestral cell type for both oocyte and spermatocyte development. Recently, a large number of techniques have been developed to differentiate human primordial germ cells (hPGCLCs) from human induced pluripotent stem cells (hiPSCs) (Mitsunage et al. 2017). Recently, new methods for monolayer hPGCLC differentiation have been developed that induce hPGCLC formation without embryoid bodies, allowing for ease of use and scalability. While many methods for hPGCLC induction exist, all suffer from large heterogeneity in hPGCLC yield depending on the cell line utilized, with many cell lines being defunct for germ cell formation. As primordial germ cells are utilized as the input cell type for in vitro ovarian and testis reconstitution, methods for high yield primordial germ cell formation are needed. In part to address these needs, a transcription factor (TF)-overexpression based technology was developed that significantly enhanced primordial germ cell yield.

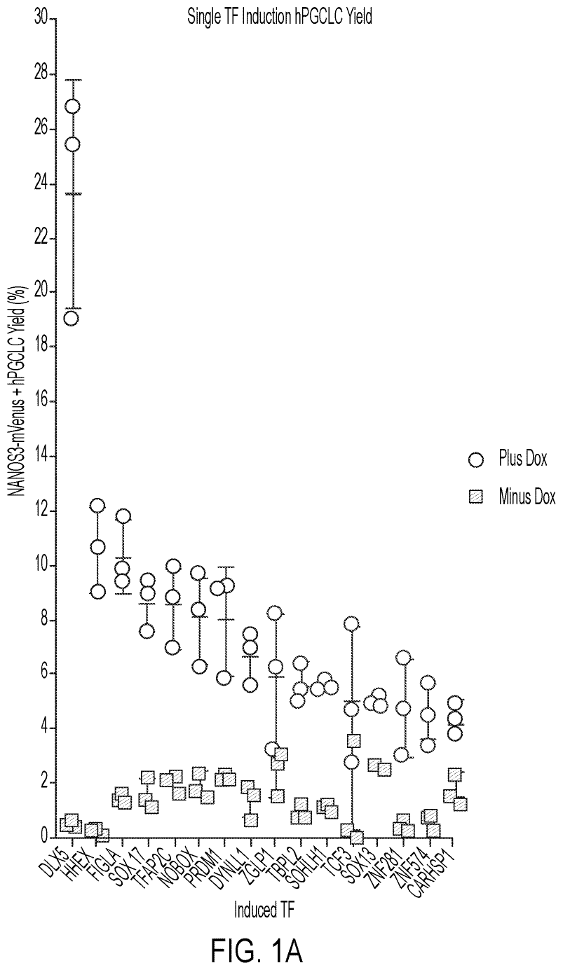

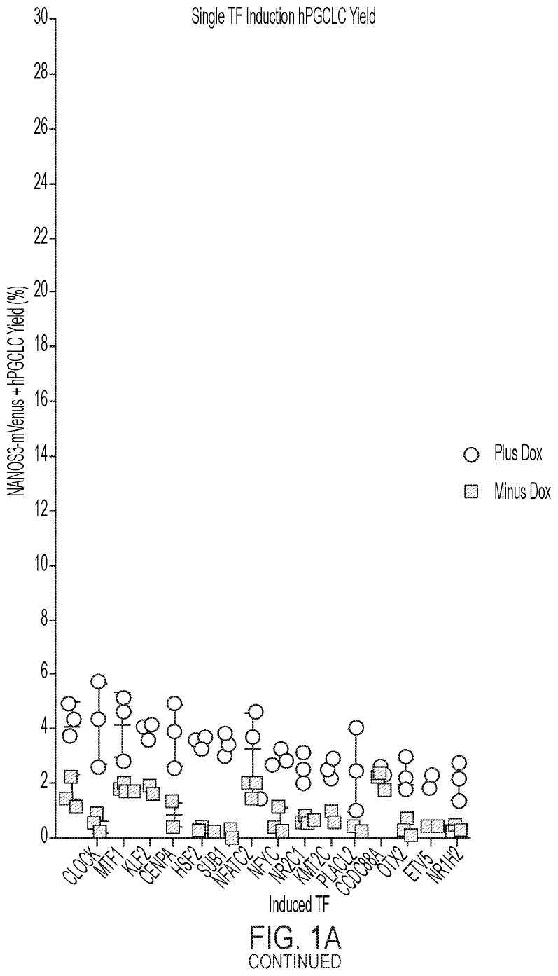

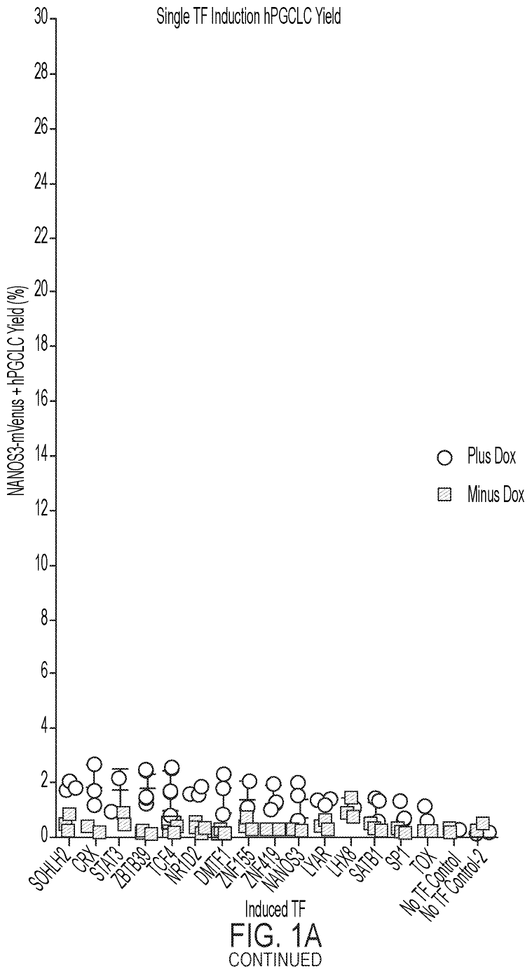

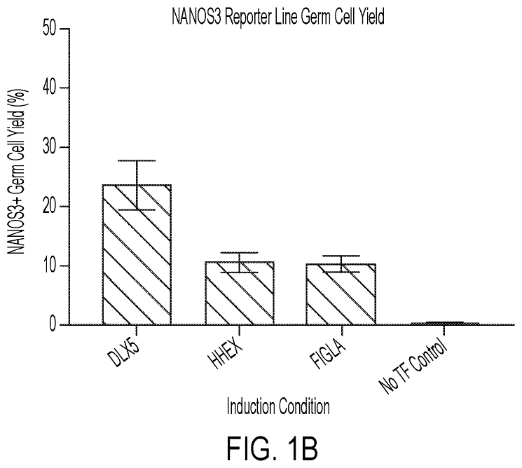

Fifty-three TFs were screened for their ability to induce robust germ cell formation from induced pluripotent stem cells (hiPSCs) (FIG. 1A). DLX5, HHEX and FIGLA were three TFs that were identified in the screen. Overexpression of these three TFs throughout the cytokine-based germ cell induction process induced a robust increase in NANOS3+ germ cell yield. Importantly, none of these three TFs was previously described in the primordial germ cell formation process, and no protocol to date has used overexpression of these three TFs to increase primordial germ cell yield.

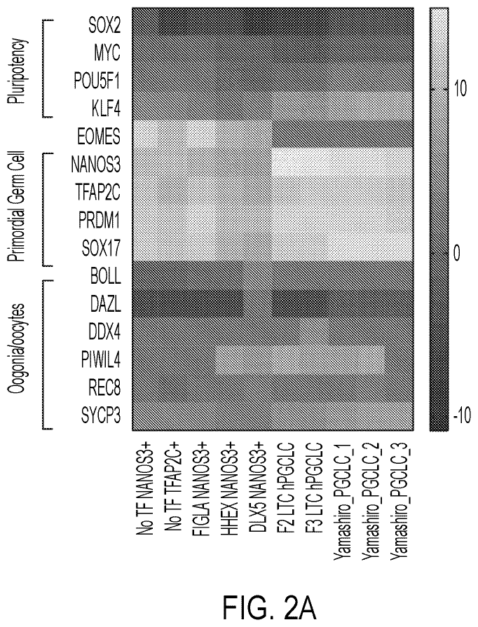

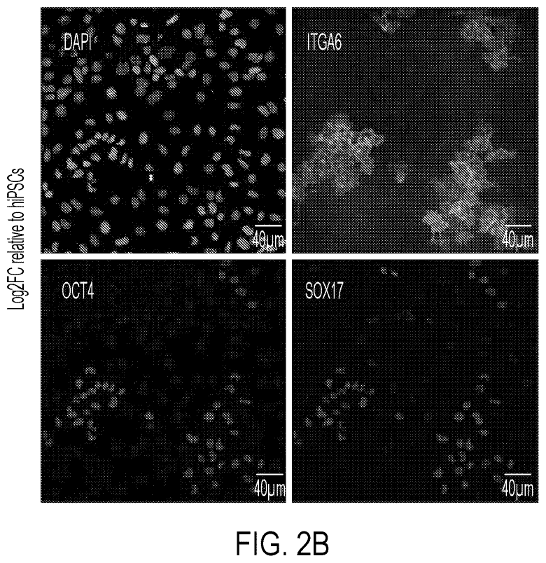

Overexpression of the three TF individually shows 35 to 80 fold increase in primordial germ cell yield, seen in the percentage of NANOS3 (a germ cell marker) cells (FIG. 1B). Of note, the germ cell line utilized was nearly germ cell incompetent, showing on average, 0.2 to 3% natural germ cell yield. With the addition of these TFs individually under doxycycline induction, hPGCLC yield was boosted to 10-35%. It was determined that the TF-induced germ cells were indeed bone fide germ cells based on their gene expression and protein expression. As can be seen, the TF-induced germ cells show the characteristic expression of SOX17, TFAP2C, and PRDM1 with upregulation of germ cell genes such as NANOS3 and downregulation of hiPSC genes such as SOX2 (FIG. 2A). In addition, the TF-induced germ cells show characteristic OCT4 and SOX17 dual positive protein expression, as can be seen through immunofluorescence imaging (FIG. 2B).

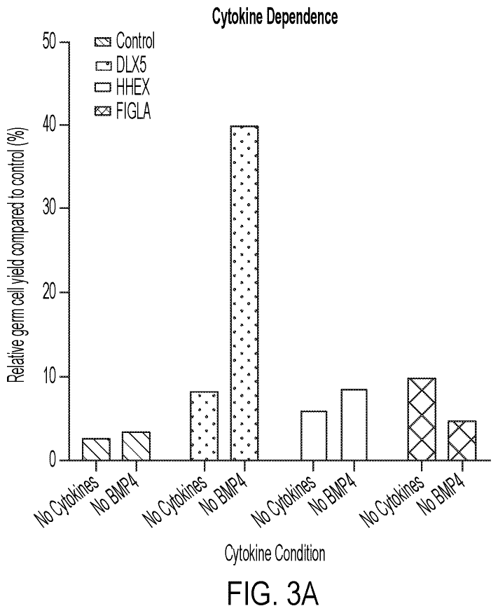

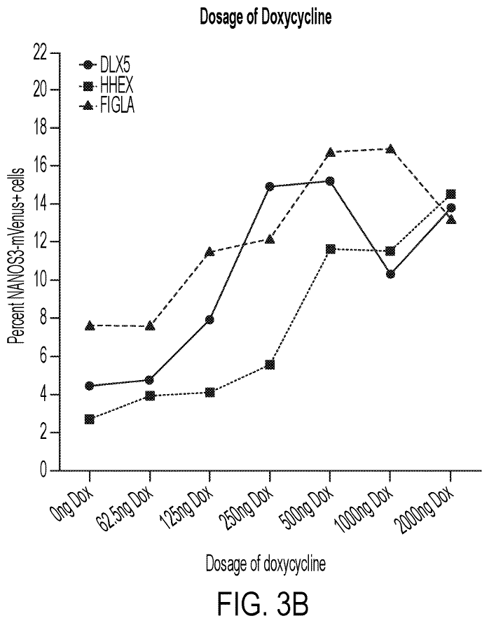

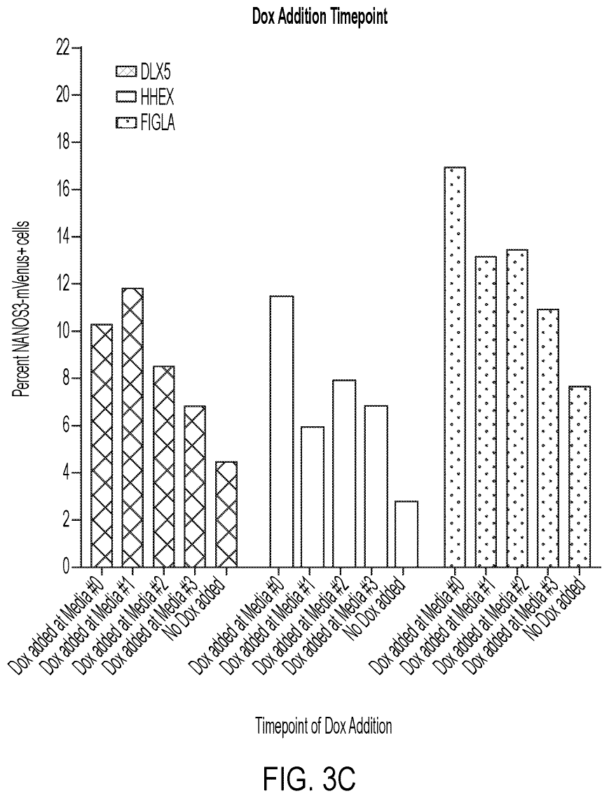

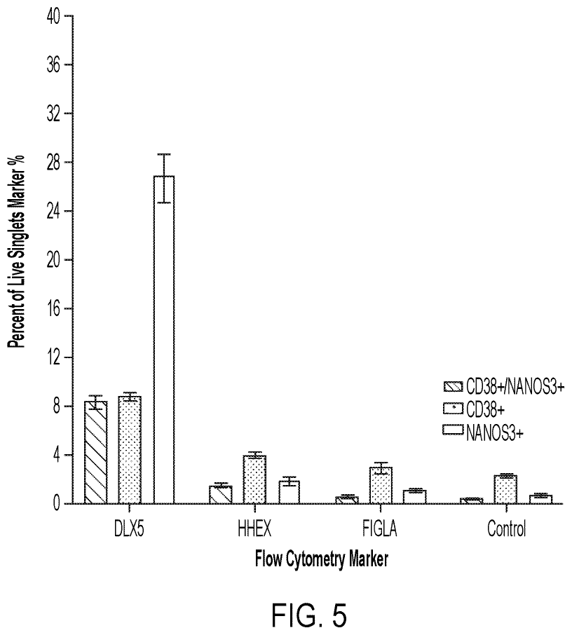

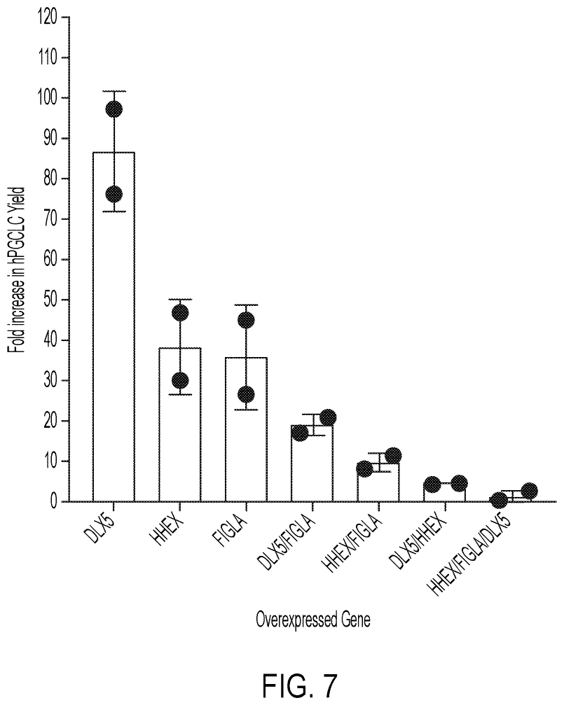

The TFs were further characterized to determine if they showed a dosage dependence, time point of induction specificity, and induction efficiency in the absence cytokines. Generally, it was determined that the germ cell yield increased with TF dosage, peaking with maximal protein expression at about 400 ng-600 ng of doxycycline (FIG. 3A). Additionally, it was determined that overexpression of the TFs throughout the germ cell formation process was generally beneficial, with HHEX being useful at the incipient mesoderm step (FIG. 3B). It was determined that DLX5 overexpression induced germ cell formation rescue at 50% compared to wildtype in the absence of BMP4, showing it could be used to help reduce or eliminate cytokine dependence on the differentiation process (FIG. 3C). Individual independent expression of FIGLA, DLX5 or HHEX is beneficial, with combinations of two or all three showing minimal additive effect or even deleterious effect (FIGS. 5 and 7).

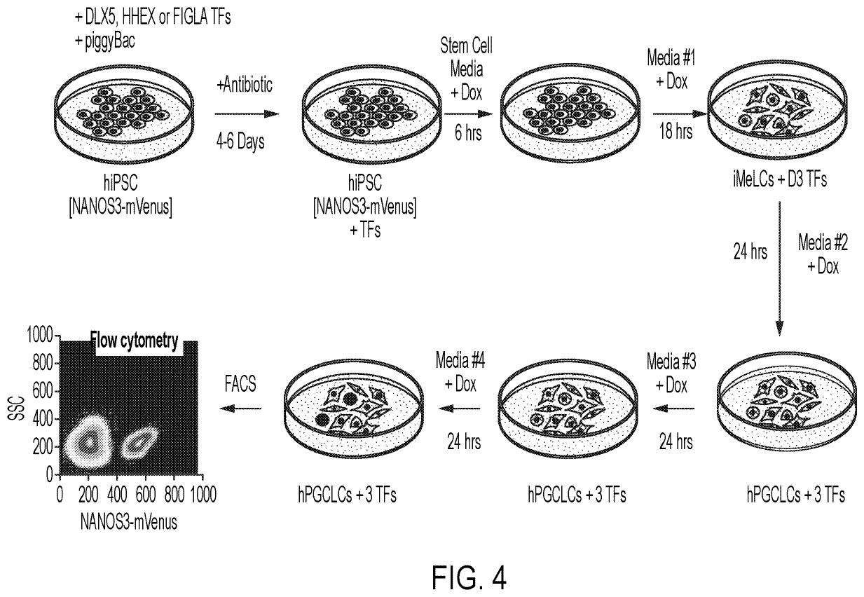

A method was designed for high yield germ cell formation from stem cells in monolayer culture conditions, which is described in more detail below and in FIG. 4 with a culture media composition described in Table 1. The TFs DLX5, HHEX, or FIGLA or all three together, were integrated to iPSCs via piggyBac or lentivirus and a purified pool was selected through antibiotic addition. For germ cell induction, 2,500 TF-containing iPSCs per cm² were seeded in mTeSR™ medium on Corning® Matrigel® Matrix for six hours. After about six hours, the media was removed and washed, and replaced by Media #1, whose components are listed in Table 1. After about 12-18 hours, Media #1 was removed and the cells were again washed, and Media #2 was added. The cells were then grown for about 24 hours, after which Media #2 was replaced by Media #3. The cells were again grown for about 24 hours, after which Media #3 was replaced by Media #4. Finally, the cells were again grown for about 24 hours, at which point primordial germ cells was harvested and isolated via FACS.

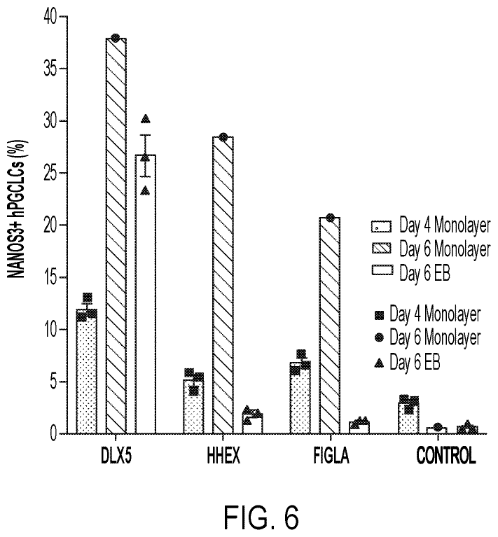

This process was entirely in monolayer, requiring no embryoid body formation and was completed in about 5-6 days. These TFs however showed inductive potential in both embryoid bodies (EBs) and monolayer (FIG. 6), making them broadly applicable to many paradigms of germ cell formation. This process is highly scalable, in which larger numbers of germ cells are easily obtained by performing the same protocol in larger plates. Without being bound by a particular theory, the process is also highly amendable to high throughput screening, capable of simple parallelization to test culture conditions, drug interactions or other developmental processes.

### Methods and Materials

iPSC Culture

iPSCs were cultured in mTESR 1 medium (Stemcell Technologies) on standard polystyrene plates coated with hESC-qualified Corning® Matrigel® Matrix. Medium was changed daily. Passaging was performed with TRYPLE (Gibco). iPSCs were treated with ˜8-12 μM Y-27632 (Ambeed) for 24 hours after each passage. Mycoplasma testing was performed by PCR every 3 months; all cells tested negative.

### TF Plasmid Construction

TF cDNAs were synthesized as full-length transcripts or obtained from the ORFeome (The ORFeome Colobration, Nat Methods. 13, 191-192 (2016)) as Gateway entry clones. These were cloned into a doxycycline-inducible PiggyBac expression plasmid (Addgene #175503) using MegaGate (Kramme et al., STAR Protoc. 2, 100907 (2021)). The final expression constructs were verified by Sanger sequencing, which also served to determine the barcode sequence for each TF.

TF Plasmid Integration into hiPSCs

Expression plasmids containing TF cDNAs under the control of a doxycycline-inducible promoter were integrated into iPSCs using PiggyBac transposase. To perform the integration, ˜50-100 fmol of TF cDNA plasmid, ˜150-250 ng PiggyBac transposase expression plasmid, and ˜100,000-200,000 iPSCs were combined in Lonza P3 buffer and electroporated using a Lonza Nucleofector 4D. After electroporation, cells were seeded in 24-well plates in mTeSR™ Plus Medium+˜8-12 μM Y-27632. Selection with the appropriate agent (typically puromycin) commenced 48 hours after electroporation and continued for ˜3-5 days. Cells were then passaged without drug selectin for ˜3 days to allow for non-integrated plasmid loss. Finally, cells were again passaged under drug selection to generate a pure, selected integrant pool. Presence and approximate copy number of integrated TF plasmids was confirmed by qPCR on genomic DNA. For oogonia, pools of hiPSCs were utilized, not single cell selected clones. Average copy number was 8-10.

### Protocol for Primordial Germ Cell Induction Via TF Overexpression

hiPSCs containing integrated TF expression plasmids were cultured in mTeSR 1 medium on Matrigel. For induction in monolayer, hiPSCs are disassociated to single cells using Accutase and seeded on Matrigel or vitronectin XF coated plates at a density of 2,500-3,000 cells per cm² in mTeSR™ media+˜8-10 μM Y-27632 and ˜0.5-3 μg/ml doxycycline for about 6 hours. Media was then removed and washed with dPBS and replaced with aRB27 media #1 (see components list in detailed protocol). After about 12-18 hours of induction, media #1 is removed and washed with dPBS and replaced by media #2. After about 24 hours, media #2 is removed and replaced by media #3. After about 24 hours, media #3 is replaced with media #4. hPGCLCs were harvested for use after about 24 hours in media #4 or additionally after about two further days of culture in media #4 (at day 6 of the protocol). hPGCLCs were isolated via NANOS3 reporter expression, CD38 cell surface expression, combinations of both or EPCAM/ITGA6 dual positive cell surface markers. hPGCLCs can additionally be generated via embryoid formation through methods established in Yamashiro et al. Science, 362(6412), 356-360, Kobayashi et al., Stem Cell Reports, 9(3), 999-1015, Stem Cell Reports, 9(3), 999-1015, Murase et al., The EMBO Journal, 1-25, 2020, and Mitsunaga et al., Proceedings of the National Academy of Sciences of the United States of America, 114(46), E9913-E9922 with similar efficiency compared to monolayer protocols.

TABLE 1
Media compositions used in Example 1

| aRB27 Media | Media #1 | Media #2 | Media #3 | Media #4 |
|---|---|---|---|---|
| Advanced | aRB27 Media | aRB27 Media | aRB27 Media | aRB27 Media |
| RPMI | | | | |
| B27 Minus or | Doxycycline | Doxycycline | Doxycycline | Doxycycline |
| Plus Vitamin A | (~0.5-3 μl/mL) | (~0.5-3 μl/mL) | (~0.5-3 μl/mL) | (~0.5-3 μl/mL) |
| (100X) | | | | |
| GlutaMAX ™ | Activin A | XAV939 | XAV939 | XAV939 |
| Supplement | (~50-150 ng/mL) | (~0.5-3 μM) | (~0.5-3 μM) | (~0.5-3 μM) |
| (100X) | | | | |
| Non-essential | Chiron99021 | hBMP4 | SCF | hBMP4 |
| amino acids | (~2-4 μM) | (~35-45 ng/mL) | (~50-150 ng/mL) | (~35-45 ng/mL) |
| (NEAA) (100X) | | | | |
| Primocin | PD173074 | | EGF | SCF |
| (100X)* | (~50-150 nM) | | (~25-75 ng/mL) | (~50-150 ng/mL) |
| Y-27632 | | | | EGF |
| (~8-12 μM) | | | | (~25-75 ng/mL) |

*Optional antibiotic

TABLE 2
Cytokines used in Example 1

| | | Product | | |
|---|---|---|---|---|
| Cytokines | Supplier | Number | Lot Number | Buffer |
| Activin A | Peprotech | 120-14P | #1117605 | 0.1% BSA in water |
| BMP4 | R&D | 314-BP-010 | #BEM12220081 | 0.1% BSA in 4 mM HCl |
| | Systems | | | |
| EGF | Peprotech | AF-100-15-1MG | #0820AFC05 | 5% trehalose (or 0.1% |
| | | | | BSA) in water |
| SCF | Peprotech | 300-07-250UG | #111834-1 | 0.1% BSA in water |

TABLE 3
Inhibitors used in Example 1

| Inhibitors | Supplier | Lot Number | Solvent |
|---|---|---|---|
| CHIR99021 | Axon | 12 | 66% H2O, |
| (hydrochloride) | Medchem | | 33% DMSO |
| PD173074 | Selleck | S126404 | DMSO |
| XAV939 | Tocris | 5B/251235 | DMSO |
| Y-27632 | Ambeed | Various | H2O |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A pluripotent stem cell (PSC) comprising: an engineered polynucleotide comprising an open reading frame encoding a protein selected from DLX5, HHEX, and FIGLA.

2. The PSC of claim 1, wherein the PSC comprises the engineered polynucleotide comprising an open reading frame encoding DLX5.

3. The PSC of claim 1, wherein the PSC comprises the engineered polynucleotide comprising an open reading frame encoding HHEX.

4. The PSC of claim 1, wherein the PSC comprises the engineered polynucleotide comprising an open reading frame encoding FIGLA.

5. The PSC of claim 1, wherein the PSC expresses or overexpresses: DLX5; HHEX; FIGLA; DLX5 and HHEX; DLX5 and FIGLA; HHEX and FIGLA; or DLX5, HHEX, and FIGLA.

6. The PSC of claim 1, wherein the open reading frame of the engineered polynucleotide is operably linked to a heterologous promoter.

7. The PSC of claim 6, wherein the heterologous promoter is an inducible promoter.

8. A pluripotent stem cell (PSC) comprising: a protein selected from DLX5, HHEX, and FIGLA, wherein the protein is overexpressed.

9. The PSC of claim 8, wherein the PSC expresses or overexpresses: DLX5; HHEX; FIGLA; DLX5 and HHEX; DLX5 and FIGLA; HHEX and FIGLA; or DLX5, HHEX, and FIGLA.

10. The PSC of claim 1, wherein the PSC is a human PSC.

11. The PSC of claim 1, wherein the PSC is an induced PSC (iPSC).

12. The PSC of claim 1, wherein the PSC comprises 1-20, optionally 8-10, copies of the engineered polynucleotide comprising the open reading frame encoding the protein selected from DLX5, HHEX, and FIGLA.

13. A composition comprising: a population of the PSC of claim 1.

14. The composition of claim 13, wherein the population comprises at least 2500/cm² of the PSC.

15. A method, comprising: culturing, in culture media, a population of pluripotent stem cells (PSCs) to produce an expanded population of PSCs; and expressing in PSCs of the expanded population a protein selected from DLX5, HHEX, and FIGLA to produce PGCLCs.

16. The method of claim 15, wherein the PSCs of the expanded population comprise an engineered polynucleotide comprising an open reading frame encoding DLX5.

17. The method of claim 15, wherein the PSCs of the expanded population comprise an engineered polynucleotide comprising an open reading frame encoding HHEX.

18. The method of claim 15, wherein the PSCs of the expanded population comprise an engineered polynucleotide comprising an open reading frame encoding FIGLA.

19. The method of claim 15, wherein the open reading frame of the engineered polynucleotide is operably linked to a heterologous promoter.

20. The method of claim 15, wherein the heterologous promoter is an inducible promoter.

21. The method of claim 15, wherein the population comprises 1×10²-1×10⁷ PSCs.

22. The method of claim 15, wherein the population of PSCs is cultured for about 3-5 days.

23. The method of claim 22, wherein the population of PSCs is cultured for about 4 days.

24. The method of claim 15, wherein the PGCLCs are NANOS3⁺, SOX17⁺, TFAP2C⁺, PRDM1⁺, OCT4⁺, CD38⁺, EPCAM⁺, ITGA6⁺, and/or SOX2⁻ PGCLCs.

25. A method comprising:
(a) delivering to pluripotent stem cells (PSCs) an engineered polynucleotide comprising an inducible promoter operably linked to an open reading frame encoding a protein selected from DLX5, HHEX, and FIGLA;
(b) culturing the PSCs in feeder-free, serum-free culture media to produce an expanded population of PSCs; and
(c) culturing PSCs of the expanded population in a series of induction media comprising an inducing agent to produce NANOS3⁺, SOX17⁺, TFAP2C⁺, PRDM1⁺, OCT4⁺, CD38⁺, EPCAM⁺, ITGA6⁺, and/or SOX2⁻ PGCLCs.

26. The method of claim 25, wherein the engineered polynucleotide is a transposon and the delivering further comprises delivering a transposase to the PSCs.

27. The method of claim 25, wherein the inducible promoter is a chemically-inducible promoter, optionally a doxycycline-inducible promoter.

28. The method of claim 25, wherein the feeder-free, serum-free culture media of (b) comprises a solubilized basement membrane preparation extracted from the Engelbreth-Holm-Swarm (EHS) mouse sarcoma.

29. The method of claim 28, wherein the solubilized basement membrane preparation comprises extracellular matrix (ECM) proteins and growth factors.

30. The method of claim 29, wherein the ECM proteins are selected from Laminin, Collagen IV, heparan sulfate proteoglycans, and entactin/nidogen.

31. The method of claim 25, wherein the feeder-free, serum-free culture media of (b) comprises growth factors selected from recombinant human basic fibroblast growth factor (rh bFGF) and recombinant human transforming growth factor β (rh TGFβ).

32. The method of claim 25, wherein the culturing of (b) is for about 6 to about 24 hours.

33. The method of claim 25, wherein the PSCs of the expanded population of (c) are cultured at a density of about 2,000 cells/cm² to about 3,000 cells/cm².

34. The method of claim 25, wherein the culturing of (c) comprises culturing the PSCs is a first induction media, culturing the PSCs in a second induction media, culturing the PSCs in a third induction media, and culturing the PSCs in a fourth induction media.

35. The method of claim 34, wherein the first induction media comprises one or more of B-27, L-alanyl-L-glutamine, an inducing agent (e.g., doxycycline), Activin A, a glycogen synthase kinase (GSK) 3 inhibitor, and a selective FGFR1 and FGFR3 inhibitor.

36. The method of claim 34, wherein the second induction media comprises one or more of B-27, an inducing agent (e.g., doxycycline), a small molecule inhibitor of tankyrase (TNKS), and a human bone morphogenic protein 4 (hBMP4).

37. The method of claim 34, wherein the third induction media comprises one or more of B-27, an inducing agent (e.g., doxycycline), a small molecule inhibitor of tankyrase, stem cell factor (SCF), and epidermal growth factor (EGF).

38. The method of claim 34, wherein the fourth induction media comprises one or more of B-27, an inducing agent (e.g., doxycycline), a small molecule inhibitor of tankyrase, hBMP4, SCF, and EGF.

39. A primordial germ cell-like cell produced by the method of claim 25.
